(19) United States
(12) Patent Application Publication
(10) Pub. No.: US 2002/0165428 A1
(43) Pub. Date: Nov. 7, 2002

(54) Personal hygiene apparatus and method

(76) Inventors: Pratima Jain, Marlboro, NJ (US); Sandhya S. Jain, Marlboro, NJ (US); Sugandh S. Jain, Marlboro, NJ (US)

Correspondence Address: Mr. Walter J. Tencza Jr., Suite 3, 10 Station Place, Metuchen, NJ, 08840, US

(21) Appl. No.: 09/846,441

(22) Filed: May 1, 2001

(51) Int. Cl. A61F 2/02

(52) U.S. Cl. 600/32

(57) ABSTRACT

The present invention discloses in an apparatus which suppresses the sound of the releasing of intestinal gasses (farts) and thus avoiding the embarrassment and ridicule that one suffers in the public company of others. The apparatus can be a very thin fibrous material such as cotton or polyester. When inserted it stays inside the anus sphincter. The sphincter stays in contracted position so no feces or excretion can escape without voluntarily loosening the anus sphincter. However, the intestinal gas can pass through the fibrous material without making any sound or noise. An apparatus for this use is disclosed comprising first, second, third, and fourth sides forming a pyramid, or a pyramid shaped device, wherein each side is comprised of a fibrous material. The pyramid may have a first end and a second end and the pyramid may be open at one end and closed at the other end. The first and second sides may be opposite one another and may be the same size. The third and fourth sides may be opposite one another and may be the same size. The sizes of the first and second sides may differ from the sizes of the third and fourth sides. Each side may be triangular shaped and have a height of about ⅝ of an inch. The fibrous material may be, for example, cotton or polyester. A method of inserting a pyramid shaped device comprised of a fibrous material into an individual's anus sphincter is also disclosed. The pyramid shaped device may be inserted so that none of the pyramid shaped device lies outside of the individual's anus.

### FIELD OF THE INVENTION

This present invention relates to apparatus and methods for personal hygiene.

### BACKGROUND OF THE INVENTION

Various personal hygiene products and methods are known.

### SUMMARY OF THE INVENTION

The present invention discloses in one embodiment an apparatus which suppresses the sound of the releasing of intestinal gasses (farts) and thus avoiding the embarrassment and ridicule that one suffers in the public company of others. The apparatus can be a very thin fibrous material such as cotton or polyester “plug”. When inserted, it stays inside the anus passed the sphincter. The sphincter stays in contracted position so no feces or excretion can escape without voluntarily loosening the anus sphincter. However, the intestinal gas can pass through the fibrous material without making any sound or noise.

The present invention in one embodiment discloses an apparatus comprising first, second, third, and fourth sides forming a pyramid, pyramid like shaped, or wedge shaped device, wherein each side is comprised of a fibrous material. The apparatus may be porous. Alternatively, the pyramid like shaped device (or wedge shaped device) may have a first end and a second end and the pyramid may be open at one end and closed at the other end. The first and second sides may be opposite one another and may be the same size. The third and fourth sides may be opposite one another and may be the same size. The sizes of the first and second sides may differ from the sizes of the third and fourth sides. Each side may be triangular shaped and have a height of about ⅝ of an inch. The fibrous material may be, for example, cotton or polyester.

An apparatus in accordance with another embodiment of the present invention or a variation may be shaped like a wedge or an unequal side pyramid. Whether the apparatus is shaped exactly like a pyramid or shaped as a wedge or unequal side pyramid, the apparatus may be porous to allow air to escape.

The present invention in one embodiment also discloses inserting a pyramid shaped device comprised of a fibrous material into an individual's anus sphincter. The pyramid shaped device may be inserted so that none of the pyramid shaped device lies outside of the individual's anus. Alternatively, a pointed end of the pyramid like shaped apparatus (or wedge shaped apparatus) may pass the anus sphincter and a part or most the pyramid like apparatus may lie outside of the anus hole.

### BRIEF DESCRIPTION OF THE DRAWINGS

FIG. 1 shows a perspective view of an apparatus for suppressing or reducing the sound of intestinal gasses (farts) in accordance with an embodiment of the present invention;

FIG. 2 shows a front planar view of the apparatus of FIG. 1;

FIG. 3 shows a side view of the apparatus of FIG. 1;

FIG. 4 shows a top planar view of the apparatus of FIG. 1;

FIG. 5 shows the apparatus of FIG. 1 inserted into an individual's anus sphincter;

FIG. 6 shows a perspective view of a second apparatus for suppressing or reducing the sound of intestinal gasses in accordance with a second embodiment of the present invention; and

FIG. 7 shows the apparatus of FIG. 6 inserted into an individual's anus sphincter so that a portion of the apparatus lies outside the anus.

### DETAILED DESCRIPTION OF THE DRAWINGS

FIG. 1 shows a perspective view of an apparatus 10 for suppressing or reducing the sound of intestinal gasses (farts) in accordance with an embodiment of the present invention. The apparatus 10 has a pyramidal wedge shape. The apparatus 10 may not be exactly pyramidal shaped but may instead be wedge shaped or be a pyramid with unequal sides. The apparatus 10 includes outer surfaces 12a, 14a, 16a, and 18a, and inner surfaces 12b, 14b, 16b, and 18b of sides 12, 14, 16, and 18. The sides 12, 14, 16, and 18 need not be equal in size. Each of the surfaces 12a-18a, and 12b-18b are shaped like triangles. Inner surfaces 12b, 14b, 16b, and 18b are opposite their surface counterpart 12a, 14a, 16a, and 18a. The apparatus 10 has a pointed closed end 20a and an end 20b having an opening 22.

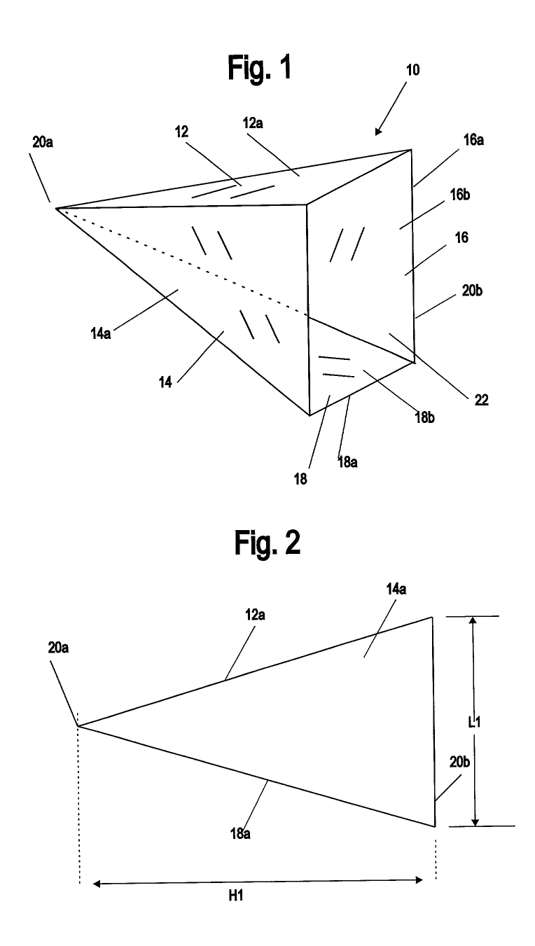

FIG. 2 shows a front planar view of the apparatus 10 of FIG. 1. Surface 14a is shown in FIG. 2.

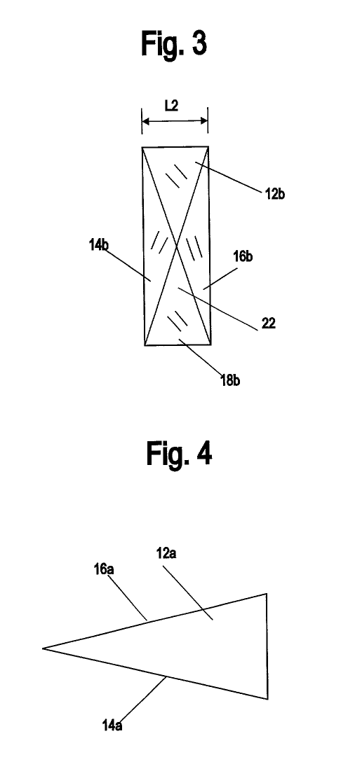

FIG. 3 shows a side view of the apparatus 10 of FIG. 1. FIG. 3 shows a view looking into opening 22 and also shows the joining of the inner surfaces 12b, 14b, 16b, and 18b.

FIG. 4 shows a top planar view of the apparatus 10 of FIG. 1. Outer surface 12a is particularly shown in FIG. 4.

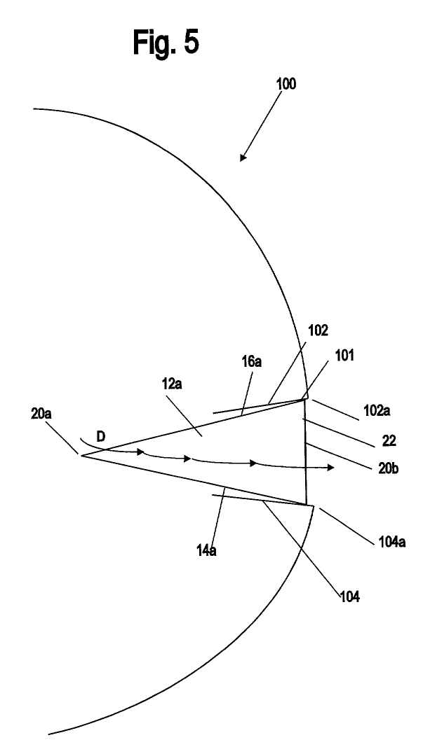

FIG. 5 shows the apparatus 10 of FIG. 1 inserted into an individual's anus 100 which includes an anus sphincter 101 having walls 102 and 104, having outside edges 102a and 104a. The apparatus 10 is inserted into the anus sphincter 101 so that the outer surfaces 16a and 14a keep the anus sphincter 101 open, i.e. prevent wall 102 from meeting with wall 104. This allows intestinal gas to pass through the apparatus 10 from inside the anus 100. The line D with arrows shows the direction of flow of intestinal gas from the individual's body and out the anus sphincter 101 from the area near end 20a out the opening 22 to the area near end 20b. Although the apparatus 10 is shown entirely inserted into the anus sphincter 101 in FIG. 5, it may be better to allow at least part of the apparatus 10 to stick out of the anus sphincter 101. Only the pointed end 20a of the apparatus 10 needs to be inside the anus sphincter 101.

The height of the apparatus 10 from the end 20a to the end 20b, shown in FIG. 2, may be H1 which may be ⅝ of an inch and this may also be the height of each of the four triangular sides 12, 14, 16, and 18. The length L2 of the base of surface 14a shown in FIG. 2 may be ½/inch. The length, L2 of the base of surface 12b shown in FIG. 3 may be 7/32 of an inch.

The apparatus 10 may be comprised of fibrous material such as cotton or polyester fibers compressed to give the apparatus 10 shape for easy insertion and handling. Each side 12, 14, 16, and 18 may be made of the fibrous material such as cotton or polyester fibers. The triangular shape of the apparatus 10 is for smooth and easy insertion into the anus sphincter 101 and comfort in the inserted position. The length of the apparatus 10 is such that the apparatus 10 may be made to not stick out the anus 100 as is shown in FIG. 5. Alternatively, the length of the apparatus 10 is such that the pointed end 20a lies pass the anus sphincter 101 and some of the apparatus 10 sticks out of the anus hole outside of the outside edges 102a and 104a.

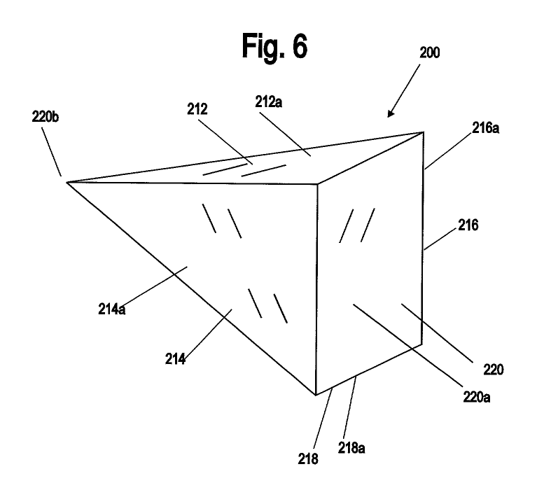

FIG. 6 shows a perspective view of a second apparatus 200 for suppressing or reducing the sound of intestinal gasses in accordance with a second embodiment of the present invention. The apparatus 200 is similar to the apparatus 10 shown in FIG. 1. However, the apparatus 200 is not hollow like the apparatus 10. The apparatus 200 is porous and may be made of a fibrous material such as cotton or polyester.

The apparatus 200 is comprised of sides 212, 214, 216, 218, and 220 having surfaces 212a, 214a, 216a, 218a, and 220a respectively. The apparatus 200 has a pointed end 220b. An individual may insert apparatus 200 in the manner shown from apparatus 10 in FIG. 5. Air can escape from all sides (212, 214, 216, 218, and 220) in the apparatus 200.

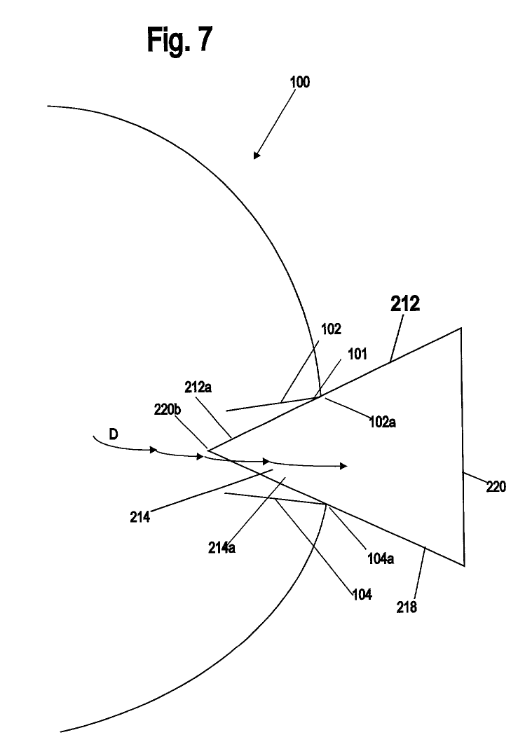

An individual does not need to insert the apparatus 10 or the apparatus 200 all the way in as shown in FIG. 5. Only the pointed end, (such as end 200b in FIG. 6) needs to pass the sphincter of the anus hole. It is better in some circumstances that a major part of the apparatus 10 or 200 sticks out of the anus sphincter 101 as shown for example for apparatus 200 by FIG. 7. Because the material of apparatus 10 or 200 is typically cotton/polyester, the individual will not even feel the apparatus 10 or 200. A portion of the apparatus 200 in FIG. 7 lies outside the edges 102a and 104a of the walls 102 and 104, respectively of the anus sphincter 101.

I claim:

1. An apparatus comprising:
first, second, third, and fourth sides forming a pyramid, each side comprised of a fibrous material.

2. The apparatus of claim 1 wherein
the pyramid has a first end and a second end and the pyramid is open at one end and closed at the other end.

3. The apparatus of claim 1 wherein
the first and second sides are opposite one another and are the same size;
the third and fourth sides are opposite one another and are the same size;
and wherein the sizes of the first and second sides differs from the sizes of the third and fourth sides.

4. The apparatus of claim 1 wherein
each side is triangular shaped and has a height of about ⅝ of an inch.

5. The apparatus of claim 1 wherein
the fibrous material is cotton.

6. The apparatus of claim 1 wherein
the fibrous material is polyester.

7. A method comprised of the step of:
inserting a pyramid shaped device comprised of a fibrous material into an individual's anus sphincter.

8. The method of claim 7 wherein
the pyramid shaped device is inserted so that none of the pyramid shaped device lies outside of the individual's anus.

9. The method of claim 7 wherein
the pyramid shaped device is comprised of a plurality of triangular sides and each triangular side has a height of about ⅝ of an inch.

10. The method of claim 8 wherein
the pyramid shaped device is comprised of a plurality of triangular sides and each triangular side has a height of about ⅝ of an inch.

11. The method of claim 7 wherein
the pyramid shaped device has a first end and a second end and the pyramid shaped device is open at one end and closed at the other end.

12. The method of claim 7 wherein
the pyramid shaped device has first, second, third, and fourth sides;
the first and second sides are opposite one another and are the same size;
the third and fourth sides are opposite one another and are the same size;
and wherein the sizes of the first and second sides differs from the sizes of the third and fourth sides.

13. The method of claim 7 wherein
the pyramid shaped device has first, second, third, and fourth sides; and
each side is triangular shaped and has a height of about ⅝ of an inch.

14. The method of claim 7 wherein
the fibrous material is cotton.

15. The method of claim 7 wherein
the fibrous material is polyester.

16. A method comprising the steps of:
inserting in an individual's anus a plug which allows intestinal gasses to escape without making a sound.
